(19) United States
(12) Patent Application Publication
(10) Pub. No.: US 2018/0243731 A1
(43) Pub. Date: Aug. 30, 2018

(54) ALKYLAROMATIC CONVERSION CATALYST

(71) Applicant: SHELL OIL COMPANY, Houston, TX (US)

(72) Inventors: Yuriy YANSON, Amsterdam (NL); Hong-Xin LI, Lansdale, PA (US); Anton PETUSHKOV, Newtown, PA (US)

(21) Appl. No.: 15/752,963

(22) PCT Filed: Aug. 16, 2016
(86) PCT No.: PCT/US2016/047125

§ 371 Date: Feb. 15, 2018

(30) Foreign Application Priority Data
Aug. 18, 2015 (EP) 15181342.5

(51) Int. Cl. B01J 29/44; B01J 29/46; B01J 29/48; B01J 21/08; B01J 35/10; C07C 4/18; C07C 15/04

(52) U.S. Cl. CPC B01J 29/44; C07C 2529/48; B01J 29/48; B01J 21/08; B01J 35/1038; B01J 35/1042; B01J 35/1061; C07C 4/18; C07C 15/04; B01J 2229/20; B01J 2229/40; C07C 2521/08; C07C 2529/44; C07C 2529/46; B01J 29/46

(57) ABSTRACT

Alkylaromatic conversion catalyst which comprises a) a carrier which comprises of from 20 to 70 wt % of a refractory oxide binder, of from 30 to 80 wt % of ZSM-5 having a mesopore volume of from 0.1 to 1.0 ml/g, a crystallite size of from 3 to 100 nm and a silica to alumina molar ratio in the range of from 20 to 200, all percentages being on the basis of total catalyst; b) an amount of from 0.001 to 5 wt % of one or more metals chosen from the group consisting of Groups 6, 9 and 10; and c) optionally a metal chosen from Group 14 in an amount up to 0.5 wt %, on the basis of total catalyst, and a process for the preparation of such catalyst.

The present invention relates to an alkylaromatic conversion catalyst, its preparation, and its use in ethylbenzene dealkylation.

### BACKGROUND OF THE INVENTION

Ethylbenzene is one of the aromatic hydrocarbons that can be obtained from naphtha pyrolysis or reformate. Reformate is an aromatic product obtained by the catalyzed conversion of straight-run hydrocarbons boiling in the 70 to 190° C. range, such as straight-run naphtha. The catalysts used for the production of reformate are often platinum-on-alumina catalysts. The reformate feedstock itself is obtained by fractionation or distillation of crude petroleum oil, its composition varying depending on the source of the crude oil, but generally having a low aromatics content. On conversion to reformate, the aromatics content is considerably increased and the resulting hydrocarbon mixture becomes highly desirable as a source of valuable chemical intermediates and as a component for gasoline. The principle components are a group of aromatics often referred to as BTX: benzene, toluene and the xylenes, including ethylbenzene. Other components may be present such as their hydrogenated homologues, e.g. cyclohexane.

Of the BTX group the most valuable components are benzene and the xylenes, and therefore BTX is often subjected to processing to increase the proportion of those two aromatics: hydrodealkylation of toluene to benzene and toluene disproportionation to benzene and xylenes. Within the xylenes, para-xylene is the most useful commodity and xylene isomerisation or transalkylation processes have been developed to increase the proportion of para-xylene.

A further process that the gasoline producer can utilize is the hydrodealkylation of ethylbenzene to benzene.

Generally, the gasoline producer will isolate BTX from the reformate stream, and then subject the BTX stream to xylene isomerisation with the aim of maximising the para-xylene component. Xylene isomerisation is a catalytic process. Some catalysts used in this process have the ability not just to isomerise xylenes but also simultaneously to dealkylate the ethylbenzene component. Normally the para-xylene is then separated out to leave benzene, toluene (unless toluene conversion processes have already been applied) and the remaining mixed xylenes, including ethylbenzene. This BTX stream can either be converted by transalkylation to increase the yield of xylenes by contacting with a heavier hydrocarbon stream or can be converted by dealkylation to selectively eliminate ethylbenzene and to increase the yield of benzene, while allowing the xylenes to reach equilibrium concentrations. The latter process is the subject of the present invention.

In ethylbenzene dealkylation at this latter stage of BTX treatment, it is a primary concern to ensure not just a high degree of conversion to benzene but also to avoid xylene loss. Xylenes may typically be lost due to transalkylation, e.g. between benzene and xylene to give toluene, or by addition of hydrogen to form, for example, alkenes or alkanes.

It is therefore the aim of the present invention to provide a catalyst that will convert ethylbenzene to benzene with a high selectivity and preferably further only will cause limited xylene loss.

Simultaneous xylene isomerisation to the equilibrium concentration of para-xylene is also desirable as this will increase the amount of para-xylene in the product. It would be especially advantageous if such alkylaromatic conversion process could be carried out at relatively high weight hourly space velocities.

### SUMMARY OF THE INVENTION

The present invention provides an alkylaromatic conversion catalyst which comprises a) a carrier which comprises of from 20 to 70 wt % of a refractory oxide binder; of from 30 to 80 wt % of ZSM-5 having a mesopore volume of from 0.1 to 1.0 ml/g, a crystallite size of from 3 to 100 nm and a silica to alumina molar ratio in the range of from 20 to 200, all percentages being on the basis of total catalyst; b) an amount of from 0.001 to 5 wt % of one or more metals chosen from the group consisting of Groups 6, 9 and 10; and c) optionally a metal chosen from Group 14 in an amount up to 0.5 wt %, on the basis of total catalyst.

Furthermore, the present invention provides a process for the preparation of such catalyst, which comprises combining of from 20 to 70 wt % of a refractory oxide binder, of from 30 to 80 wt % of ZMS-5 having a mesopore volume of from 0.1 to 1.0 ml/g, a crystallite size of from 3 to 100 nm and a silica to alumina molar ratio in the range of from 20 to 200, and less than 10 wt % of an optional other component, shaping the resulting mixture, if desired, and compositing the mixture with an amount of from 0.001 to 5 wt % of one or more metals chosen from the group consisting of Groups 6, 9 and 10 and optionally a metal chosen from Group 14 in an amount up to 0.5 wt %, all percentages being on the basis of total catalyst.

All weight amounts, as the term is used in relation with the catalyst composition or the catalyst preparation, are based on dry amounts. Any water and other solvent present in the starting compounds is disregarded.

The mesopores, as the term is used herein, are those pores of the ZSM-5 having a pore diameter in the range of from 50 to 350 angstroms (Å). These are measured according to ASTM D4365-13.

The macropores are the pores of the catalyst having a pore diameter greater than 350 Å, more specifically of from 350 to 2000 Å. These are measured according to ASTM D4284.

The micropores, as the term is used herein, are those pores of the catalyst having a pore diameter less than 50 angstroms (Å). These are measured according to ASTM D4222-03.

The crystallite size is measured by Transmission Electron Microscopy (TEM) with the average based on the number average.

Groups 6, 9, 10 and 14 are as defined in the IUPAC Periodic Table of Elements dated 1 May 2013.

The weight amounts of metal are calculated as amount of metal on total weight of catalyst.

Also provided is an ethylbenzene dealkylation process which comprises contacting in the presence of hydrogen a feedstock which comprises ethylbenzene with a catalyst according to the present invention or a catalyst as prepared by a process according to the present invention.

### DETAILED DESCRIPTION OF THE INVENTION

Catalyst of the present invention has been found to have a high alkylaromatic conversion activity in that a lower operating temperature is required. It was especially surprising that additionally the xylenes in the product can have a relatively high para-xylene content as relatively small ZSM-5 particles provide more surface area without steric hindrance thereby potentially allowing for side-reactions. Furthermore, it was found that the catalyst can have a relatively high benzene selectivity combined with reduced xylene losses at a given ethylbenzene conversion.

The ZSM-5 for use in the carrier of the present invention preferably has a mesopore volume of at least 0.10 ml/g, more specifically at least 0.15 ml/g, most specifically at least 0.20 ml/g. The mesopore volume preferably is at most 0.90 ml/g, more specifically at most 0.80 ml/g, more specifically at most 0.70 ml/g, more specifically at most 0.60 ml/g, more specifically at most 0.50 ml/g, most specifically at most 0.40 ml/g.

The macropore volume of the catalyst preferably is at least 0.3 ml/g, more specifically at least 0.4 ml/g, most specifically at least 0.5 ml/g. The macropore volume of the catalyst preferably is at most 1.5 ml/g, more specifically at most 1.0 ml/g.

The micropore volume of the catalyst preferably is at least 0.01 ml/g, more specifically at least 0.02 ml/g. The micropore volume of the catalyst preferably is at most 0.09 ml/g, more specifically at most 0.08 ml/g, most specifically at most 0.06 ml/g.

The crystallite size of the ZSM-5 for use in the present invention preferably is at least 3 nm, more specifically at least 5 nm, more specifically at least 10 nm, more specifically at least 20 nm. The crystallite size preferably is at most 90 nm, more specifically at most 80 nm, more specifically at most 70 nm, more specifically at most 60 nm, more specifically at most 50 nm, most specifically at most 40 nm.

The silica to alumina molar ratio of the ZSM-5 preferably is at least 20, more specifically at least 30, more specifically at least 40, most specifically at least 50. This ratio preferably is at most 180, more specifically at most 150, more specifically at most 120, most specifically at most 110.

The present invention most preferably uses ZSM-5 which is commercially available from Zeolyst under the trade name ZD13008.

The binder is a refractory oxide, more preferably a refractory oxide selected from the group consisting of silica, zirconia and titania.

Most preferably, silica is used as a binder in the catalyst composition of the present invention. It may be naturally occurring silica or may be in the form of a gelatinous precipitate, sol or gel. The form of silica is not limited and the silica may be in any of its various forms: crystalline silica, vitreous silica or amorphous silica. The term amorphous silica encompasses the wet process types, including precipitated silicas and silica gels, or pyrogenic or fumed silicas. Silica sols or colloidal silicas are non-settling dispersions of amorphous silicas in a liquid, usually water, typically stabilized by anions, cations, or non-ionic materials.

The silica binder preferably is a mixture of two silica types, most preferably a mixture of a powder form silica and a silica sol. Conveniently powder form silica has a B.E.T. surface area in the range of from 50 to 1000 m²/g; and a mean particle size in the range of from 2 nm to 200 micron m, preferably in the range of from 2 to 100 micron m, more preferably 2 to 60 micron m, especially 2 to 10 micron m as measured by ASTM C 690-1992 or ISO 8130-1. A very suitable powder form silica material is Sipernat 50, a white silica powder having predominantly spherical particles, available from Degussa (Sipernat is a trade name). A very suitable silica sol is that sold under the trade name of Bindzil by Eka Chemicals. Where the mixture comprises powder form silica and a silica sol, then the two components may be present in a weight ratio of powder form to sol form in the range of from 1:1 to 10:1, preferably 2:1 to 5:1, more preferably from 2:1 to 3:1. The binder may also consist essentially of just the powder form silica.

Where solely a powder form of silica is used as a binder in the catalyst composition of the present invention, preferably a small particulate form is utilized, which has a mean particle size in the range of from 2 to 10 micron as measured by ASTM C 690-1992. An additional improvement in carrier strength is found with such materials. A very suitable small particulate form is that available from Degussa under the trade name Sipernat 500LS.

Preferably the silica component is used as pure silica and not in combination with other refractory oxide components. It is most preferred that the silica and indeed the carrier, is essentially free of any other inorganic oxide binder material, and especially is free of alumina. At most only a maximum of 2 wt % alumina, based on the total refractory oxide binder, is present.

The carrier of the present invention preferably comprises of from 20 to 70% wt of binder in combination with of from 30 to 80% wt of ZSM-5, more specifically of from 25 to 60% wt of binder in combination with of from 40 to 75% wt of ZSM-5, more specifically of from 25 to 65% wt of binder in combination with of from 30 to 75% wt of ZSM-5, most specifically 30 to 50% wt of binder in combination with of from 50 to 70% wt of ZSM-5.

The mixture of zeolite and refractory oxide binder may be shaped into any convenient form such as powders, extrudates, pills and granules. Preference is given to shaping by extrusion. To prepare extrudates, commonly the pentasil zeolite will be combined with the binder, preferably silica, and if necessary a peptizing agent, and mixed to form a dough or thick paste. The peptizing agent may be any material that will change the pH of the mixture sufficiently to induce deagglomeration of the solid particles.

Peptising agents are well known and encompass organic and inorganic acids, such as nitric acid, and alkaline materials such as ammonia, ammonium hydroxide, alkali metal hydroxides, preferably sodium hydroxide and potassium hydroxide, alkali earth hydroxides and organic amines, e.g. methylamine and ethylamine.

Ammonia is a preferred peptizing agent and may be provided in any suitable form, for example via an ammonia precursor. Examples of ammonia precursors are ammonium hydroxide and urea. It is also possible for the ammonia to be present as part of the silica component, particularly where a silica sol is used, though additional ammonia may still be needed to impart the appropriate pH change. The amount of ammonia present during extrusion has been found to affect the pore structure of the extrudates which may provide advantageous properties. Suitably the amount of ammonia present during extrusion may be in the range of from 0 to 5 wt % based on the total dry mixture, preferably 0 to 3 wt %, more preferably 0 to 1.9 wt %, on dry basis.

The ZSM-5 present in the catalyst has properties very similar to those of the ZSM-5 used as starting compound in the preparation. Therefore, the preferences for catalyst components and ratios also apply to the components used in preparing the catalyst.

The catalyst of the present invention comprises of from 0.001 to 5 wt % of one or more metals chosen from the group consisting of Groups 6, 9 and 10 and optionally a metal chosen from Group 14 in an amount up to 0.5 wt %, on the basis of total catalyst. Preferably, the metal of Group 6, 9 or 10 is chosen from the group consisting of tungsten, molybdenum, cobalt, nickel, palladium and platinum while the metal of Group 14 is chosen from lead and tin. Most preferably, the catalyst comprises of from 0.001 to 0.1% wt of platinum and/or palladium, most preferably platinum, based on amount of metal on total amount of catalyst. The amount preferably is from 0.01 to 0.05% wt. Additionally, such catalyst can contain one or more further catalytically active compounds, most preferably tin.

The catalyst of the present invention may be prepared using standard techniques for combining the zeolite, binder such as silica, and optional other carrier components; shaping; compositing with the metals components; and any subsequent useful process steps such as drying, calcining, and reducing.

The metals emplacement onto the formed carrier may be by methods usual in the art. The metals can be deposited onto the carrier materials prior to shaping, but it is preferred to deposit them onto a shaped carrier.

Pore volume impregnation of the metals from a metal salt solution is a very suitable method of metals emplacement onto a shaped carrier. The metal salt solutions may have a pH in the range of from 1 to 12. The platinum salts that may conveniently be used are chloroplatinic acid and ammonium stabilized platinum salts. If tin is present, the tin preferably is added as a salt selected from the group consisting of stannous (II) chloride, stannic (IV) chloride, stannous sulphate, and stannous acetate.

If different metals are deposited on the carrier, the metals may be impregnated onto the shaped carrier either sequentially or simultaneously. Where simultaneous impregnation is utilized the metal salts used must be compatible and not hinder the deposition of the metals. It has been found useful to utilize a complexing or chelating agent in a combined platinum/tin salt solution to prevent unwanted metals precipitation. Examples of suitable complexing agents are EDTA (ethylenediamine tetraacetic acid), and derivatives thereof, HEDTA (N-(2-hydroxyethyl) ethylenediamine-N,N′,N′-triacetic acid), EGTA (ethylene glycol-bis(2-aminoethylether)-N,N,N′,N′-tetraacetic acid), DTPA (diethylene tridiamine pentaacetic acid), and NTA (nitrilotriacetic acid). Where EDTA is used, it is conveniently used in a molar ratio to tin of from 0.1 to 3, especially 1 to 2.

After shaping of the carrier, and also after metals impregnation, the carrier/catalyst is suitably dried, and calcined. Drying temperatures are suitably 50 to 200° C.; drying times are suitably from 0.5 to 5 hours. Calcination temperatures are very suitably in the range of from 200 to 800° C., preferably 300 to 600° C. For calcination of the carrier, a relatively short time period is required, for example 0.5 to 3 hours. For calcination of the catalyst composition, it may be necessary to employ controlled temperature ramping at a low rate of heating to ensure the optimum dispersion of the metals: such calcination may require from 5 to 20 hours.

Prior to use, it is generally necessary to ensure that the metals on the catalyst composition are in metallic (and not oxidic) form. Accordingly, it is useful to subject the composition to reducing conditions, which are, for example, heating in a reducing atmosphere, such as in hydrogen optionally diluted with an inert gas, or mixture of inert gases, such as nitrogen and carbon dioxide, at a temperature in the range of from 150 to 600° C. for from 0.5 to 5 hours.

The catalyst composition of the invention finds especial use in the selective dealkylation of ethylbenzene.

The ethylbenzene feedstock most suitably originates directly from a reforming unit or naphtha pyrolysis unit or is the effluent of a xylene isomerisation unit. Such feedstock usually comprises hydrocarbons containing of from 7 to 9 carbon atoms, and in particular one or more of o-xylene, m-xylene, p-xylene, toluene, and benzene in addition to ethylbenzene. Generally the amount of ethylbenzene in the feedstock is in the range of from 0.1 to 50 wt % and the total xylene content is typically at least 20 wt %, based on total amount of hydrocarbon feed.

Typically the xylenes will not be in a thermodynamic equilibrium, and the content of p-xylene will accordingly be lower than that of the other isomers.

The feedstock is contacted with the catalyst in the presence of hydrogen. This may be carried out in a fixed bed system, a moving bed system, or a fluidized bed system. Such systems may be operated continuously or in batch fashion. Preference is given to continuous operation in a fixed bed system. The catalyst may be used in one reactor or in several separate reactors in series or operated in a swing system to ensure continuous operation during catalyst change-out.

The process is suitably carried out at a temperature in the range of from 300 to 500° C., a pressure in the range of from 0.1 to 50 bar (10 to 5,000 kPa), using a weight hourly space velocity of in the range of from 0.5 to 20 g feed/g catalyst/hour. A partial pressure of hydrogen in the range of from 0.05 to 30 bar (5 to 3,000 kPa) is generally used. The feed to hydrogen molar ratio is in the range of from 0.5 to 100, generally from 1 to 10 mol/mol. As the catalyst of the present invention is especially suitable for use in high weight hourly space velocity processes, the preferred operating conditions comprise a weight hourly space velocity of in the range of from 7 to 17 g feed/g catalyst/hour, more specifically of from 8 to 14 g feed/g catalyst/hour, an overall pressure of from 5 to 25 bar (500 to 2,500 kPa), more specifically 8 to 15 bar (800 to 1,500 kPa) and a feed to hydrogen molar ratio in the range of from 1 to 5 mol/mol.

The present invention will now be illustrated by the following Examples.

### EXAMPLES

In the Examples and where mentioned elsewhere hereinabove, the following test methods are applicable: Flat plate crush strength: ASTM D 6175.

Micropore volume: ASTM D4222-03.
Mesopore volume: ASTM D4365-13.
Macropore volume: ASTM D4284-07.
Crystallite size: number average as measured by Transmission Electron Microscopy (TEM).
BET surface area: ASTM D4222-03.

### Example 1

A carrier was prepared from ZSM-5 having a mesopore volume of 0.015 ml/g, a crystallite size of 200 nm and a silica to alumina molar ratio of 80. This ZSM-5 is commercially available from Zeolyst as CBV 8014G.

The zeolite powder was mixed with a low sodium grade silica (Sipernat 50 from Degussa), and an ammonium stabilized commercially available silica sol (sold under the trade name Bindzil by Eka Chemicals), and extruded using 1.5 wt % of ammonium hydroxide solution (containing 25 wt % ammonia) on dry basis to give a carrier comprised of 60 wt % zeolite, 26.7 wt % Sipernat 50 and 13.3 wt % silica sol on dry basis.

The green extrudates were dried and calcined at about 550° C. for 1 hour to achieve sufficient strength for industrial application.

The resulting carrier contained 40% wt of silica binder and 60% wt of zeolite, based on dry weight.

The carrier was pore volume impregnated with a platinum containing solution having a pH below 2. The solution was prepared from H2PtCl6. The concentration of metal was such as to provide a final catalyst having a concentration of 0.025 wt %, based on total catalyst. Once the impregnation was completed, the catalyst was dried at 125° C. for 3 hours, and subsequently calcined in a two-step calcination program aiming at 480° C. with a sufficient low ramping rate to achieve adequate dispersion of the metallic phase. The total calcination procedure lasted 12 hours. This comparative catalyst is hereinafter referred to as Catalyst 1.

The catalyst obtained had an average flat plate crush strength of 85 N/cm, a BET surface area of 358 m²/g, a micropore volume of 0.091 ml/g and a macropore volume of 0.555 ml/g.

### Example 2

A carrier was prepared from ZSM-5 having a mesopore volume of 0.29 ml/g, a crystallite size of 28 nm and a silica to alumina molar ratio of 80. This ZSM-5 is commercially available from Zeolyst as ZD13008.

The zeolite powder was mixed with a low sodium grade silica (Sipernat 50 from Degussa), and an ammonium stabilized commercially available silica sol (sold under the trade name Bindzil by Eka Chemicals), and extruded using 1.5 wt % of ammonium hydroxide solution (containing 25 wt % ammonia) on dry basis to give a carrier comprised of 60 wt % zeolite, 26.7 wt % Sipernat 50 and 13.3 wt % silica sol on dry basis.

The green extrudates were dried and calcined at about 550° C. for 1 hour to achieve sufficient strength for industrial application.

The resulting carrier contained 40% wt of silica binder and 60% wt of zeolite, based on dry weight.

The carrier was pore volume impregnated with a platinum containing solution having a pH below 2. The solution was prepared from H2PtCl6. The concentration of metal was such as to provide a final catalyst having a concentration of 0.025 wt %, based on total catalyst. Once the impregnation was completed, the catalyst was dried at 125° C. for 3 hours, and subsequently calcined in a two-step calcination program aiming at 480° C. with a sufficient low ramping rate to achieve adequate dispersion of the metallic phase. The total calcination procedure lasted 12 hours. This catalyst is hereinafter referred to as Catalyst 2.

The catalyst obtained had an average flat plate crush strength of 76 N/cm, a BET surface area of 430 m²/g, a micropore volume of 0.039 ml/g and a macropore volume of 0.649 ml/g.

### Example 3

Catalysts 1 and 2 were subjected to a catalytic test that mimics typical industrial application conditions for ethylbenzene dealkylation. This activity test uses an industrial feed of which the composition is summarized in Table 1.

TABLE 1
Composition of the feed used in the activity testing
Feed composition

| | |
|---|---|
| EB wt % | 15.30 |
| pX wt % | 2.71 |
| oX wt % | 15.62 |
| mX wt % | 63.26 |
| toluene wt % | 0.28 |
| benzene wt % | 0.02 |
| C7-C8-naphthenes wt % | 2.81 |
| C9+ aromatics wt % | 0.00 |
| Total wt % | 100.00 |
| C8 aromatics sum wt % | 96.89 |
| EB in C 8 aromatics feed wt % | 15.79 |
| pX in xylenes in feed wt % | 3.32 |
| oX in xylenes in feed wt % | 19.14 |
| mX in xylenes in feed wt % | 77.53 |

The activity test is performed once the catalyst is in its reduced state, which is achieved by exposing the dried and calcined catalyst to atmospheric hydrogen (>99% purity) at 450° C. for 1 hour.

After reduction the reactor is pressurized without a cooling step, and the feed is introduced. This step contributes to enhanced catalyst aging, and therefore allows comparison of the catalytic performance at stable operation.

The catalytic datapoints are collected at a condition that exaggerates the potential negative operational effects. Therefore, the performance is measured not at the ideal industrial operating conditions but at those that allow a better differentiation of the various performance parameters used to evaluate catalysts in this application.

In the present case, a weight hourly space velocity of 12 g feed/g catalyst/hour, a hydrogen to feed ratio of 2.5 mol·mol⁻¹ and a total system pressure of 1.2 MPa was used. The temperature was varied between 340 and 380° C. to achieve the required conversion for easier comparison.

The performance characteristics including the product obtained are shown in Table 2 below.

Ethylbenzene conversion (EB conversion) is the weight percent of ethylbenzene converted by the catalyst into benzene and ethylene, or other molecules. It is defined as wt % ethylbenzene in feed minutes wt % ethylbenzene in product divided by wt % ethylbenzene in feed times 100%.

PXate is a measure for the degree to which the xylene reaction mixture has reached equilibrium for para-xylene. It is defined as follows:

$$PXate = \frac{\% w PX in Xylenes in product - \% w PX in Xylenes in feed}{\begin{matrix}\% w PX in Xylenes at equilibrium - \\ \% w PX in Xylenes in feed\end{matrix}} \times 100 \%$$

where PX stands for para-xylene.

Xylene loss is calculated as wt % xylenes in feed minus wt % xylenes in product divided by wt % xylenes in feed times 100%.

TABLE 2

| | Catalyst 1 | Catalyst 2 |
|---|---|---|
| Reactor temperature ° C. | 378 | 361 |
| C1-C6 wt % | 2.31 | 2.30 |
| C7-C8-naphthenes wt % | 2.44 | 2.39 |
| EB wt % | 7.63 | 7.63 |
| pX wt % | 18.50 | 19.29 |
| oX wt % | 17.27 | 17.99 |
| mX wt % | 44.39 | 43.03 |
| toluene wt % | 1.15 | 0.87 |
| benzene wt % | 5.17 | 5.22 |
| C9+ aromatics wt % | 1.14 | 1.28 |
| EB conversion, wt % | 50 | 50 |
| pX in xylenes, wt % | 23.08 | 24.02 |
| pXate, % | 97.5 | 101.9 |
| Xylene loss, wt % | 1.76 | 1.57 |

The above experimental results show that catalyst according to the present invention does not only require a lower temperature to achieve the required performance but also has a product which contains a relatively large amount of para-xylene as shown by the content of the para-xylene in the product obtained (pX in xylenes). The para-xylene content of the xylene reaction mixture is even higher than the equilibrium value. Besides high ethylbenzene conversion activity and high para-xylene content of the product, the catalyst according to the invention gave less xylene loss than comparative catalyst 1.

1. The alkylaromatic conversion catalyst which comprises
a) a carrier which comprises of from 20 to 70 wt % of a refractory oxide binder; of from 30 to 80 wt % of ZSM-5 having a mesopore volume of from 0.1 to 1.0 ml/g, a crystallite size of from 3 to 100 nm and a silica to alumina molar ratio in the range of from 20 to 200, all percentages being on the basis of total catalyst;
b) an amount of from 0.001 to 5 wt % of one or more metals chosen from the group consisting of Groups 6, 9 and 10; and c) optionally a metal chosen from Group 14 in an amount up to 0.5 wt %, on the basis of total catalyst.

2. An alkylaromatic conversion catalyst as claimed in claim 1, wherein the refractory oxide binder is chosen from the group consisting of silica, zirconia and titania.

3. An alkylaromatic conversion catalyst as claimed in claim 1, wherein the carrier is composed of in the range of from 25 to 60 wt % silica, and in the range of from 40 to 75 wt % ZSM-5.

4. An alkylaromatic conversion catalyst as claimed in claim 1, wherein the ZSM-5 has a mesopore volume of from 0.20 to 0.90 ml/g and a crystallite size of from 5 to 80 nm.

5. An alkylaromatic conversion catalyst as claimed in claim 1, wherein the ZSM-5 has a SAR in the range of from 20 to 150.

6. An alkylaromatic conversion catalyst as claimed in claim 1, wherein the binder is present in an amount of from 30 to 50% wt and ZSM-5 is present in an amount in the range of from 50 to 70 wt %.

7. An alkylaromatic conversion catalyst as claimed in claim 1 comprising platinum and/or palladium in an amount in the range of from 0.001 to 0.1 wt % and optionally tin in an amount up to 0.5 wt %.

8. A process for the preparation of an alkylaromatic conversion catalyst as claimed in claim 1, which process comprises combining of from 20 to 70 wt % of a refractory oxide binder, of from 30 to 80 wt % of ZMS-5 having a mesopore volume of from 0.1 to 1.0 ml/g, a crystallite size of from 3 to 100 nm and a silica to alumina molar ratio in the range of from 20 to 200, and less than 10 wt % of an optional other component, shaping the resulting mixture, if desired, and compositing the mixture with an amount of from 0.001 to 5 wt % of one or more metals chosen from the group consisting of Groups 6, 9 and 10 and optionally a metal chosen from Group 14 in an amount up to 0.5 wt %, all percentages being on the basis of total catalyst.

9. An ethylbenzene dealkylation process which comprises contacting in the presence of hydrogen a feedstock which comprises ethylbenzene, with an alkylaromatic conversion catalyst as claimed in claim 1.

10. An ethylbenzene dealkylation process according to claim 9, in which process the weight hourly space velocity is of from 7 to 15 g feed/g catalyst/hour, the pressure is in the range of from 5 to 25 bar and the hydrogen/hydrocarbon molar ratio is of from 1 to 5.
